(19) United States
(12) Patent Application Publication
(10) Pub. No.: US 2026/0224793 A1
(43) Pub. Date: Aug. 6, 2026

(54) Body Liquid Drainage System

(71) Applicant: C. R. Bard, Inc., Franklin Lakes, NJ (US)

(72) Inventor: Prasad Ventrapragada, Marietta, GA (US)

(21) Appl. No.: 19/152,415

(22) PCT Filed: Feb. 1, 2023
(86) PCT No.: PCT/US23/12095

§ 371 Date: Jul. 30, 2025

(51) Int. Cl. A61M 1/00; A61M 25/00

(52) U.S. Cl. CPC A61M 1/743; A61M 1/60; A61M 1/73; A61M 1/742; A61M 25/0017; A61M 2205/18; A61M 2205/3341; A61M 2205/3365; A61M 2205/502; A61M 2205/7536

(57) ABSTRACT

A drainage system for draining a liquid from a patient including a drainage tube defining fluidly coupled between a catheter and collection container. A pinch valve and vent-port valve are coupled with the drainage tube adjacent the catheter. A vacuum pump fluidly coupled with the collection container defines a vacuum therein. Logic of a system module activates and deactivates the pinch valve, vent-port valve, and vacuum pump to perform a purging process of the drainage tube. The logic may initiate and discontinue the purging process in response to signals from pressure sensors, flow meters, and a fluid detector.

### BACKGROUND

The draining of liquid (e.g., urine) from a patient may include the use of a liquid drainage system including a flexible drainage tube extending from a drainage catheter to a collection container. Typical catheters include indwelling catheters, Foley catheters, balloon catheters, peritoneal drainage catheters, or the like, and are configured to be inserted into an orifice within the body of a patient to drain a liquid therefrom. In some instances, the flexibility of the drainage tube can form sections of positive incline, also termed “dependent loops,” along the drainage tube where drainage liquid can accumulate. Liquid pooling within dependent loops can cause various complications. For example, urine pooling can be a source of catheter associated urinary tract infection (“CAUTI”) causing agents such as bacteria, microbes, and the like. Hospital Acquired Infections (“HAI”), such as CAUTI, are detrimental to the patient, and also incur extra costs in treating these additional complications. Embodiments disclosed herein are directed to clearing drainage liquid from dependent loops thereby, reducing patient risk.

### SUMMARY

Briefly summarized, disclosed herein is a drainage system for draining a liquid from a patient. The system, according to some embodiments, includes a drainage tube defining a proximal end and a distal end, where the drainage tube is fluidly coupled with a catheter so as to receive a drainage liquid from the catheter. A vent-port valve is coupled between the drainage tube and the catheter, and the vent-port valve transitionable between (i) a first valve state defining a flow path between the drainage tube and the catheter, and (ii) a second valve state defining a flow path between the drainage tube and the environment. A pinch valve is fluidly coupled between the vent-port valve and the catheter, where the pinch valve transitionable between (i) an open state defining a flow path between the catheter and the vent-port valve and (ii) a closed state defining a closure of the flow path between the catheter and the vent-port valve. A collection container is coupled with the drainage tube at the proximal end to collect the drainage liquid. A vacuum pump is fluidly coupled with the collection container via an air inlet tube. A system module operatively coupled with the vacuum pump and the pinch valve includes a console having a processor and a memory with logic stored thereon. The logic, when executed by the processor, causes operations of the drainage system that include (i) activating the pinch valve to transition the pinch from the open state to the closed state and activating the vacuum pump to generate a vacuum within collection container such that when the vent-port valve is transitioned from the first valve state to the second valve state, the vacuum within the collection container is released causing an airflow proximally along the drainage tube from the vent-port valve to the collection container.

In some embodiments, the vent-port valve is an electro-mechanical vent-port valve operatively coupled with the system module, and the operations include activating the vent-port valve to transition the vent-port valve from the first state to the second valve state. In some embodiments, the operations further include (i) activating the vacuum pump with the vent-port valve disposed in the first valve state and the pinch valve disposed in the closed state to cause a buildup of vacuum within the collection container and (ii) activating the vent-port valve to release the buildup of vacuum within the collection container, thereby causing a surge of airflow proximally along the drainage tube.

In some embodiments, the system further includes a pressure sensor fluidly coupled with the air inlet tube and operatively coupled with the system module, where the pressure sensor is configured to determine a vacuum level within the collection container. In some embodiments, the operations further include comparing the vacuum level with a vacuum level limit stored in the memory and adjusting a speed of the vacuum pump based on the comparison of the vacuum level with the vacuum level limit.

In some embodiments, the operations further include comparing the vacuum level with a safe vacuum limit stored in the memory and deactivating the pinch valve to transition the pinch valve from the closed state to the open state when vacuum level is below the safe vacuum limit.

In some embodiments, the system further includes a vacuum relief valve fluidly coupled with the air inlet tube, where the vacuum relief valve defines a vacuum relief limit. In such embodiments, the vacuum relief valve remains in a default closed state when the vacuum level is below the vacuum relief limit, and the vacuum relief valve self-transitions from the default closed state to an open state to relieve vacuum within the collection container when the vacuum level exceeds the vacuum relief limit.

In some embodiments, the vacuum relief valve is an electro-mechanical relief valve operatively coupled with the system module, and the operations include (i) comparing the vacuum level with a vacuum relief limit stored in the memory and (ii) activating the vacuum relief valve to transition the vacuum relief valve from the default closed state to the open state to relieve the vacuum within the collection container when the vacuum level exceeds a vacuum relief limit stored in the memory.

In some embodiments, the system further includes a first flow meter coupled in line with the air inlet tube and operatively coupled with the system module, where the first flow meter is configured to determine a first airflow rate within the inlet air tube when the vacuum pump is activated. In some embodiments, the operations further include (i) comparing the first airflow rate with a predefined first airflow rate stored in the memory and (ii) adjusting a speed of the vacuum pump based on the comparison of the first airflow rate with the predefined first airflow rate.

In some embodiments, the system further includes a second flow meter coupled in line with an air outlet tube of the vacuum pump and operatively coupled with the system module, where the second flow meter is configured to determine a second airflow rate within the outlet air tube when the vacuum pump is activated. In some embodiments, the operations further include (i) comparing the second airflow rate with a predefined second airflow rate stored in the memory and (ii) adjusting a speed of the vacuum pump based on the comparison of the second airflow rate with the predefined second airflow rate.

In some embodiments, the system further includes a fluid detector operatively coupled with the drainage tube and operatively coupled with the system module, the fluid detector configured to detect pooled liquid within the drainage tube. In some embodiments, the operations further include activating the vacuum pump in response to the fluid detector detecting pooled liquid within the drainage tube.

In some embodiments, the system further includes a charcoal filter disposed in line with outlet air tube such that air exiting the outlet tube passes through the charcoal filter.

In some embodiments, the system further includes an outlet pressure sensor fluidly coupled with the air outlet tube and operatively coupled with the system module, where the outlet pressure sensor is configured to determine an outlet pressure within the outlet tube.

In some embodiments, the operations further include (i) comparing the outlet pressure with an outlet pressure limit stored in the memory and (ii) providing a notification when the outlet pressure exceeds the outlet pressure limit.

Also disclosed herein is a method for draining liquid from a patient that, according to some embodiments, includes (i) establishing a passive flow of a drainage liquid from the patient along a drainage tube of a drainage system from a catheter to a collection container, where the drainage tube includes a pinch valve and a vent-port valve disposed in line with the drainage tube at a distal end thereof, where the pinch valve is disposed distal the vent-port valve; (ii) activating the pinch valve to close a flow path of the drainage tube between the vent-port valve and the catheter; (iii) activating the vent-port valve to define a flow path between the drainage tube and the environment; and (v) activating a vacuum pump of the drainage system, where the vacuum pump is fluidly coupled with the collection container via an air inlet tube to generate a vacuum within the collection container, thereby defining an airflow proximally along the drainage tube from the vent-port valve to the collection container.

In some embodiments of the method, activating the vent-port valve occurs after activating the vacuum pump to cause a buildup of vacuum within the collection container, such that activating the vent-port valve causes a surge of airflow proximally along the drainage tube.

In some embodiments, the method further includes determining a vacuum level within the collection container via a pressure sensor of the drainage system, where the pressure sensor is fluidly coupled with the air inlet tube. In some embodiments, the method further includes (i) comparing the vacuum level with a vacuum level limit stored in a memory of the system and (ii) adjusting a speed of the vacuum pump based on the comparison of the vacuum level with the vacuum level limit.

In some embodiments, the method further includes (i) comparing the vacuum level with a safe vacuum limit stored in the memory and (ii) deactivating the pinch valve to open the flow path of the drainage tube between the vent-port valve and the catheter when vacuum level is below the safe vacuum limit.

In some embodiments of the method, the system includes a vacuum relief valve fluidly coupled with the air inlet tube, where the vacuum relief valve defines a vacuum relief limit, and where (i) the vacuum relief valve remains in a default closed state when the vacuum level is below the vacuum relief limit and (ii) the vacuum relief valve self-transitions from the default closed state to an open state to relieve the vacuum within the collection container when the vacuum level exceeds the vacuum relief limit. In some embodiments of the method, the vacuum relief valve is an electro-mechanical relief valve, and the method further includes (i) comparing the vacuum level with a vacuum relief limit stored in the memory and (ii) activating the vacuum relief valve to transition the vacuum relief valve from the default closed state to the open state to relieve the vacuum within the collection container when the vacuum level exceeds a vacuum relief limit stored in the memory.

In some embodiments of the method, the system further includes a first flow meter coupled in line with the air inlet tube, and the method further includes determining a first airflow rate within the inlet air tube when the vacuum pump is activated.

In some embodiments, the method further includes (i) comparing the first airflow rate with a predefined first airflow rate stored in the memory and (ii) adjusting a speed of the vacuum pump based on the comparison of the first airflow rate with the predefined first airflow rate.

In some embodiments of the method, the system further includes a second flow meter coupled in line with an air outlet tube of the vacuum pump, and the method further includes determining a second airflow rate within the outlet air tube when the vacuum pump is activated. In some embodiments, the method further includes (i) comparing the second airflow rate with a predefined second airflow rate stored in the memory and (ii) adjusting a speed of the vacuum pump based on the comparison of the second airflow rate with the predefined second airflow rate.

In some embodiments of the method, the system further includes a fluid detector operatively coupled with the drainage tube, and the method further includes (i) detecting pooled liquid within the drainage tube and (ii) activating the vacuum pump in response detecting the pooled liquid within the drainage tube.

In some embodiments, the method further includes passing air exiting the outlet tube through a charcoal filter.

In some embodiments of the method, the system further includes an outlet pressure sensor fluidly coupled with the air outlet tube, and the method further includes (i) determining an outlet pressure within the outlet tube, (ii) comparing the outlet pressure with an outlet pressure limit stored in the memory, and (iii) providing a notification when the outlet pressure exceeds the outlet pressure limit.

These and other features of the concepts provided herein will become more apparent to those of skill in the art in view of the accompanying drawings and the following description, which describe particular embodiments of such concepts in greater detail.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is a schematic illustration of a liquid drainage system for draining liquid from a patient, in accordance with some embodiments disclosed herein;

FIG. 2 is a block diagram of a console of the system of FIG. 1, in accordance with some embodiments disclosed herein; and

FIG. 3 is a block diagram of a system method for draining liquid from a patient according the system of FIG. 1, in accordance with some embodiments disclosed herein.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

The phrases “connected to” and “coupled with” refer to any form of interaction between two or more entities, including mechanical, electrical, magnetic, electromagnetic, fluid, and thermal interaction. Two components may be connected or coupled with each other even though they are not in direct contact with each other. For example, two components may be coupled with each other through an intermediate component.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user. The term “fluid” as used herein may refer to either a gas or a liquid.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software, such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

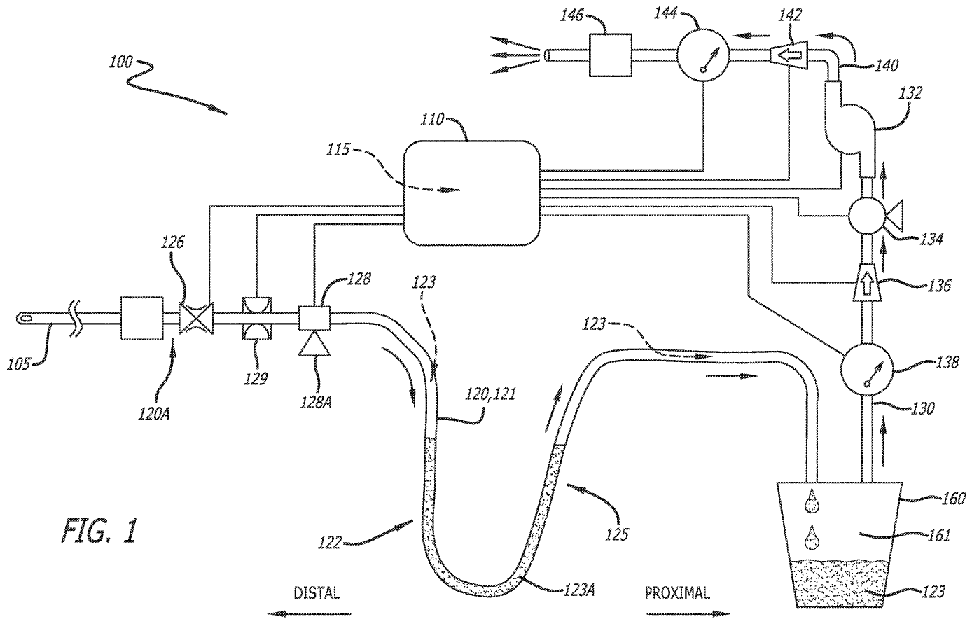

FIG. 1 illustrates an exemplary drainage system (“system”) 100, including a catheter 105, a drainage tube 120, and a collection container 160. The catheter 105 may include a urinary catheter, such as an internal catheter or an external catheter configured for use with a male or female patient. In general, the system provides a drainage pathway to transport liquid 123 from the catheter 105 to the collection container 160. The system 100 further includes a system module 110 having a console 115 including logic that generally governs the operations of the system 100 as further described below.

The drainage tube 120 including a lumen 121 extends from the catheter 105 to the collection container 160. In use, liquid 123 (e.g., urine) from the patient may flow from the catheter 105, through the drainage tube 120, and into the collection container 160. The drainage tube 120 can be formed of rubber, plastic, polymer, silicone, or similar suitable material. In the illustrated embodiment, the collection container 160 includes a rigid container. However, in other embodiments, the collection container 160 may include a semi-rigid container or a bag. The collection container 160 may generally define a closed (i.e., non-vented) container. In operation, the drainage system 100 may facilitate a passive draining process of the liquid 123 from the patient without incident. In some instances, one or more complications may arise during the passive draining process requiring corrective action as further described below.

As shown in FIG. 1, the flexibility of the drainage tube 120 can result in sections of the drainage tube 120 having one or more dependent loops 122 followed by a positive incline section 125. The positive incline section 125 can lead to collection of pooled liquid 123A liquid within the dependent loop 122 of the drainage tube 120. A dependent loop 122 may be any portion of the drainage tube 120 that is lower than a downstream portion so as to create a positive incline 125 relative to the direction of fluid flow. Dependent loops 122 can form in slack portions of the drainage tube 120. The dependent loop 122 may be a complete loop, a partial loop, or any segment of the drainage tube 120 that causes the liquid 123 to pool in the drainage tube 120.

In instances of pooling, a drainage tube clearing process (purging process), as described herein, may provide a corrective action to the pooled liquid 123A. In the illustrated embodiment, a vacuum pump 132 can provide a pressure difference across the pooled liquid 123A within the drainage tube 120 to proximally move the pooled liquid 123A proximally along the drainage tube 120. An air inlet tube 130 fluidly couples an inlet of the vacuum pump 132 to the collection container 160, and an air outlet tube 140 extends away from an outlet of the vacuum pump 132, where an open end of the air outlet tube 140 vents air to the environment 40.

The system 100 may include a number of fluidic components disposed along the drainage tube 120, such as a pinch valve 126, a vent-port valve 128, and a fluid detector 129. The pinch valve 126 is operatively coupled with the drainage tube 120 at the distal end 120A, and the vent-port valve 128 is operatively coupled with the drainage tube 120 at the distal end 120A proximal the pinch valve 126. The pinch valve 126 is generally configured to selectively allow and prevent fluid flow along the drainage tube 120 between the catheter 105 and the vent-port valve 128. The pinch valve 126 is transitionable between (i) a normally (i.e., deactivated) open state defining an open flow path between the catheter 105 and the vent-port valve 128 and (ii) a closed (i.e., activated) state defining a closure of the flow path between the catheter 105 and the vent-port valve 128. The pinch valve 126 is operatively coupled with the system module 110 so that the logic may govern the operation of the pinch valve 126. The pinch valve 126 may include reusable equipment coupled with the drainage tube 120.

The vent-port valve 128 is generally configured to selectively the drainage tube 120, i.e., selectively provide a side-port vent 128A between the lumen 121 of the drainage tube 120 and the environment 40. The vent-port valve 128 is transitionable between (i) a first valve state (closed-port state) where the lumen 121 is isolated from the environment 40 and (ii) a second valve state (open-port state) where the lumen 121 is in fluid communication with the environment 40 via the side port vent 128A, thereby allowing air to enter the drainage tube 120 via the side port vent 128A. In some embodiments, a normal (default) state of the vent-port valve 128 may be the first valve state, such that activating the vent-port valve 128 transitions the vent-port valve 128 from the first valve state to the second valve state. The vent-port valve 128 is operatively coupled with the system module 110 so that the logic may govern the operation of the vent-port valve 128. In some embodiments, the vent-port valve 128 may include a reusable equipment portion and a disposable portion of the drainage tube 120.

In some embodiments, the vent-port valve 128 may be a three way valve coupled in line with the drainage tube 120. Such a three-way valve 128 may be transitionable between (i) a first valve state defining a flow path between the drainage tube 120 and the catheter 105 and (ii) a second valve state defining a flow path between the drainage tube 120 and a side port in fluid communication with the environment 40. In some embodiments of the three-way valve, the flow path between the drainage tube 120 and the catheter 105 may be closed in the second valve state. In some embodiments, the three-way valve may include a three-way stopcock which may be manually manipulated by the clinician.

In some embodiments, the pinch valve 126 and the vent-port valve 128 may be combined to define a combination valve that provides the functionality of the pinch valve 126 and vent-port valve 128. The combination may valve includes (i) an inlet port in fluidly coupled with the catheter 105, an outlet port fluidly coupled with the collection container 160, and a side port in fluid communication with the environment 40. In some embodiments, the combination valve may be transitionable between a first valve state, a second valve state, and a third valve state. In the first valve state, the inlet port and the outlet port are in fluid communication with each other to define a flow path along the drainage tube 120 between the catheter 105 and the collection container 160, and the side port is not in fluid communication with either the inlet port or the outlet port. In the second valve state, the inlet port is not in fluid communication with the outlet port thereby closing the flow path along the drainage tube 120 between the catheter 105 and the collection container 160 consistent with the functionality of the pinch valve 126. In the second valve state, the side port remains not in fluid communication with either the inlet port or the outlet port. In the third valve state, the inlet port remains not in fluid communication with the outlet port, and the side port is disposed in fluid communication with the outlet port to define a flow path between the environment 40 and the lumen 121 of the drainage tube 120 extending toward the collection container 160.

The fluid detector 129 is operatively coupled with the drainage tube 120. The fluid detector 129 is generally configured to ascertain a state of the fluid within the drainage tube 120 at the location of the fluid detector 129. The fluid detector 129 may be configured to couple with the drainage tube 120 at any location (or any one of several locations) along the drainage tube 120. In such embodiments, the clinician may couple the fluid detector 129 at any chosen location along the drainage tube 120. In the illustrated embodiment, the fluid detector 129 is disposed between the pinch valve 126 and the vent-port valve 128. At such a location, the fluid detector 129 may indicate when the patient excretes urine. According other another embodiment, the fluid detector 129 may be coupled the drainage tube 120 along a dependent loop, e.g., the dependent loop 122, as shown so that the fluid detector 129 may detect the pooled liquid 123A along the drainage tube 120. In some embodiments, the fluid detector 129 may be configured to determine a condition of the liquid 123 extending entirely across a cross section the lumen 121 of the drainage tube 120. In other embodiments, the fluid detector 129 may be configured to detect any liquid 123 within the lumen 121. The fluid detector 129 is operatively coupled with the system module 110 so that the fluid detector 129 may provide a signal to the system module 110 for processing by the logic. The fluid detector 129 may include any technology suitable for non-invasively detecting the liquid 123 within the drainage tube 120, such as ultrasonic or optical technologies, for example. In some embodiments, the fluid detector 129 may be omitted.

The system 100 may include a number of fluidic components disposed along the air inlet tube 130, such as a vacuum relief valve 134, an inlet flow meter 136, and an inlet pressure sensor 138. In some embodiments, any of the vacuum relief valve 134, the inlet flow meter 136, or the inlet pressure sensor 138 may be omitted.

The vacuum relief valve 134 is generally configured to relieve/release vacuum from the air inlet tube 130 and, by association, the collection container 160. In some embodiments, the vacuum relief valve 134 defines a vacuum level limit so that when the vacuum within the air inlet tube 130 exceeds the vacuum level limit, the vacuum relief valve 134 allows air to enter the air inlet tube 130 to limit the vacuum, i.e., prevent the vacuum within the air inlet tube 130 from exceeding the vacuum level limit. As such, the vacuum relief valve 134 may prevent damage to the collection container 160 (e.g., crushing) and/or other components due to excessive vacuum. In some embodiments, the vacuum relief valve 134 may be operatively coupled with the system module 110 so that the logic may govern the operation of the vacuum relief valve 134. In some embodiments, the vacuum relief valve 134 may be omitted.

The inlet flow meter 136 is coupled in line with the air inlet tube 130 and is generally configured to determine an airflow rate along the air inlet tube 130. The inlet flow meter 136 is coupled with the system module 110 so that the inlet flow meter 136 may provide a signal to the system module 110 for processing by the logic.

The inlet pressure sensor 138 is operatively coupled with the air inlet tube 130 so as to determine a vacuum level within the air inlet tube 130 and collection container 160. The inlet pressure sensor 138 is coupled with the system module 110 so that the inlet pressure sensor 138 may provide a signal to the system module 110 for processing by the logic.

The vacuum pump 132 is disposed between the air inlet tube 130 and the air outlet tube 140. The vacuum pump 132 is generally configured to generate a vacuum within the collection container 160. The vacuum pump 132 is coupled with the system module 110 so that the logic my govern the operation of the vacuum pump 132, e.g., activate and deactivate the vacuum pump 132. In some embodiments, the vacuum pump 132 may include a variable speed pump so that the logic may adjust the speed of the vacuum pump, thereby adjusting the airflow rate along the air inlet tube 130 and/or the vacuum level within the collection container 160.

The system 100 may include a number of fluidic components disposed along the air outlet tube 130, such as an outlet flow meter 142, an outlet pressure sensor 144, or a charcoal filter 146. In some embodiments, any of the outlet flow meter 142, the outlet pressure sensor 144, or the charcoal filter 146 may be omitted.

The outlet flow meter 142 is coupled in line with the air outlet tube 140 and is generally configured to determine an airflow rate along the air outlet tube 140. The outlet flow meter 142 is coupled with the system module 110 so that the outlet flow meter 142 may provide a signal to the system module 110 for processing by the logic.

The outlet pressure sensor 144 is operatively coupled with the air outlet tube 140 so as to determine a positive pressure level within in the air outlet tube 140. The outlet pressure sensor 144 is coupled with the system module 110 so that the outlet pressure sensor 144 may provide a signal to the system module 110 for processing by the logic.

The charcoal filter 146 is coupled in line with the air outlet tube 140 and is generally configured to filter the air exiting the air outlet tube 140 to the environment 40. Such filtering may remove/reduce an odor of the air exiting the air outlet tube 140.

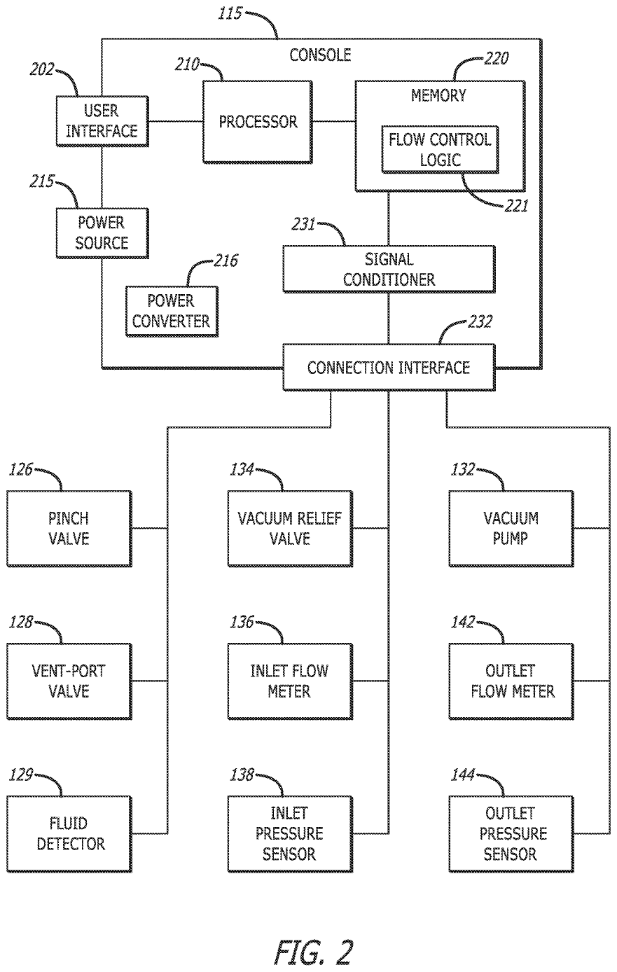

FIG. 2 illustrates a block diagram of the console 115, according to some embodiments. The console 115 is generally configured to govern the operation of the system 100. The console 115 includes a processor 210 and memory 220 (e.g., a non-transitory computer-readable medium) having flow control logic 221 stored thereon. The console 115 is powered via a power source 215, e.g., a facility power, a battery, or a combination thereof. A power converter 216 defines the specific power sources for the console 115 and components.

In some embodiments, the console 115 may include a user interface 202 configured to directly receive input from a clinician and provide output to the clinician. For example, the user interface 202 may include a number of buttons (not shown), such as an on/off button, for example. Similarly, the user interface 202 graphical user interface configured to receive input from the clinician and provide notification to the clinician.

The console 115 includes a connection interface 232 that defines operative coupling between the various components and the console 115. A signal conditioner 231 converts electrical signals from the components to digital data for processing by the processor 210 according to the flow control logic 221. The components, i.e., the pinch valve 126, the vent-port valve 128, the fluid detector 129, the vacuum relief valve 134, the inlet flow meter 136, the inlet pressure sensor 138, the vacuum pump 132, the outlet pressure sensor 142, and the outlet flow meter 144 may be each coupled with the console 115 via the connection interface 232.

The flow control logic (logic) 221 defines the operations of the system 100 including the individual components of the system 100. Some exemplary operations of the individual components are described below. The logic 221 defines the operation of the pinch valve 126. More specifically, the logic 221 may activate the pinch valve 126 to transition the pinch valve 126 from the normally open state to the closed state to isolate the catheter 105 from the drainage tube 120, or more specifically, to isolate the patient from a pressure/vacuum within the drainage tube 120. The logic 221 may also deactivate the pinch valve 126 to transition the pinch valve 126 back from the closed state to the open state to enable the liquid 123 to passively flow proximally along the drainage tube 120.

The logic 221 defines the operation of the vent-port valve 128. More specifically, the logic 221 may activate the vent-port valve 128 to transition the vent-port valve 128 from the normal/default first valve state to the second valve state to vent the lumen 121 of the drainage tube 120 to the environment 40. The logic 221 may also deactivate the vent-port valve 128 to transition the vent-port valve 128 back from the second state to the first state to close off the vent to the environment 40.

The logic 221 defines the operation of the fluid detector 129. More specifically, the logic 221 receives and processes a signal from the fluid detector 129. In some embodiments, the signal may be a binary signal indicating the presence versus absence of the liquid 123 within the drainage tube 120 at the location of the fluid detector 129. In other embodiments, the signal may be a variable signal that indicates a portion of the cross section of the lumen 121 occupied by the liquid 123.

The logic 221 defines the operation of the vacuum pump 132. More specifically, the logic 221 may activate the vacuum pump 132 to transition the vacuum pump from a normally “off” state to an “on” state to cause the vacuum pump 132 to draw air out of the collection container 160, thereby defining a vacuum within the collection container 160. The logic 221 may also deactivate the vacuum pump 132, i.e., transition the vacuum pump 132 from the “on” state to the “off” state. In some embodiments, the logic 221 may adjust a speed of the vacuum pump 132. In some embodiments, adjusting the speed of the vacuum pump 132 may correspondingly adjust a fluid flow rate along the drainage tube 120.

The logic 221 may, according to some embodiments, define the operation of the vacuum relief valve 134. More specifically, the logic 221 may activate the vacuum relief valve 134 to transition the vacuum relief valve 134 from a normally closed (i.e., a non-pressure relieving state) to a pressure relieving state, where in the pressure relieving state the vacuum relief valve 134 vents the inlet air tube 130 to the environment 40, thereby relieving vacuum from the collection container 160.

The logic 221 defines the operation of the inlet flow meter 136. More specifically, the logic 221 receives a variable signal from the inlet flow meter 136 related to an airflow rate along the air inlet tube 130. In some embodiments, the airflow rate along the air inlet tube 130 may be related to the fluid flow rate along the drainage tube 120. The logic 221 defines the operation of the outlet flow meter 142. More specifically, the logic 221 receives a variable signal from the outlet flow meter 142 related to an airflow rate along the air outlet tube 140. In some embodiments, the airflow rate along the air outlet tube 140 may be related to the airflow rate through the charcoal filter 146.

The logic 221 defines the operation of the inlet pressure sensor 138. More specifically, the logic 221 receives a variable signal from the inlet pressure sensor 138 related to the vacuum within the collection container 160. The logic 221 defines the operation of the outlet pressure sensor 144. More specifically, the logic 221 receives a variable signal from the outlet pressure sensor 144 related to the positive pressure within the outlet air tube 140.

The logic 221 may define operations of the system 100 including operations of the individual components in combination to perform a purging process of the drainage tube 120. The logic 221 may activate the vacuum pump 132, the pinch valve 126, and the vent-port valve 128 in combination to cause as airflow proximally along the drainage tube 120 from the vent port 128A to the collection container 160. The airflow exerts a force proximally on the liquid 123 disposed with the drainage tube 120 to cause the liquid 123 within the drainage tube 120 to flow along the drainage tube 120 toward the collection container 160. In some instances, the airflow may cause the pooled drainage liquid 123A to flow into the collection container 160. In some embodiments, the logic 221 may adjust the speed of the vacuum pump 132 to purge the liquid 123 more effectively from the drainage tube 120. To discontinue the purging process, the logic 221 may deactivate the vacuum pump 132, the pinch valve 126, and the vent-port valve 128, where discontinuing the purging process enables the drainage liquid 123 to flow passively along the drainage tube 120 from the catheter 105 to the collection container 160. In some embodiments, the logic 221 may perform the purging process for a defined time period, such as over a few second or over a few minutes, for example. In some embodiments, the logic 221 may initiate the purging process according to a defined schedule, and in some embodiments, the schedule may be defined by the clinician via input to the user interface 202.

In some embodiments of the purging process, the logic 221 may activate only the vacuum pump 132 and the pinch valve 126 to cause a build-up of vacuum within the collection container 160. After the build-up of vacuum, the logic 221 may activate the vent-port valve 128 to enable the built-up vacuum to cause a surge of airflow along the drainage tube 120, where the surge of airflow causes the drainage liquid 123 including the pooled drainage liquid to flow along the drainage tube 120 toward the collection container 160.

In some embodiments, the logic 221 may initiate the purging process based on a presence of the liquid 123 detected within the drainage tube 120 via the fluid detector 129. For example, the logic 221 may initiate the purging process upon detection of any liquid 123 disposed within the drainage tube 120, such as at the beginning of urine excretion by the patient. By way of another example, the logic 221 may initiate the purging process when the fluid detector 129 detects the presence of the liquid 123 within the drain tube 120 for an extended period of time as may be indicative of the pooled liquid 123A within the drainage tube 120. In similar fashion, the logic 221 may discontinue the purging process when the fluid detector 129 detects the absence the liquid 123 along the drainage tube 120 or a portion thereof.

In some embodiments, the logic 221 may operate the vacuum pump 132 to define the vacuum within the collection container 160 according to a predefined vacuum level by determining the vacuum level via the inlet pressure sensor 138. For example, the logic 221 may compare the determined vacuum level with the predefined vacuum level stored in the memory 220, and the logic 221 may alter the operation (e.g., activate, deactivate, or adjust the speed) of the vacuum pump 132 based on the comparison of the determined vacuum level with the predefined vacuum level. In some embodiments, the logic 221 may control the speed of the vacuum pump to maintain the determined vacuum level at or near the predefined valve vacuum level.

In some embodiments, the logic 221 may deactivate the pinch valve 126 according to a defined safe vacuum limit. In other words, the logic 221 may maintain activating the pinch valve 126 until the vacuum, as determined by the inlet pressure sensor 138, is below the safe vacuum limit. In some embodiments, the logic 221 may compare the vacuum level determined by the inlet pressure sensor 138 with a safe vacuum limit stored in the memory 220 and deactivate the pinch valve 126 only when the determined vacuum level is below the safe vacuum limit.

In some embodiments, the logic 221 may deactivate the vacuum pump 132 according to a maximum safe vacuum level. As the collection container 160 and other components may be configured operate to within a defined vacuum range, the logic 221 may be configured to prevent damage to the collection container 160 by preventing the vacuum from exceeding a maximum safe limit. For example, the logic 221 compare the determined vacuum level with a maximum safe vacuum limit stored in the memory 220, and based on the comparison, the logic 221 may deactivate the vacuum pump 132 when the determined vacuum level exceeds the maximum safe vacuum limit.

In some embodiments, the logic 221 may alter the operation of the vacuum pump 132 according to a determined airflow rate defined by the vacuum pump 132, where the airflow rate may be determined by one or both of the inlet flow meter 136 or the outlet flow meter 142. For example, the logic 221 may compare an inlet airflow rate determined by the inlet flow meter 136 with a defined inlet airflow rate stored in the memory 220, and based on the comparison, the logic 221 may alter the operation of the vacuum pump 132, e.g., the logic 221 may (i) increase the speed of the vacuum pump 132 when the determined inlet airflow rate is below the defined inlet airflow rate and/or (ii) decrease the speed of the vacuum pump 132 when the determined inlet airflow rate exceeds the defined inlet airflow rate. By way of another example, the logic 221 may compare an outlet airflow rate determined by the outlet flow meter 142 with a defined outlet airflow rate stored in the memory 220, and based on the comparison, the logic 221 may alter the operation of the vacuum pump 132, e.g., the logic 221 may (i) increase the speed of the vacuum pump 132 when the determined outlet airflow rate is below the defined outlet airflow rate and/or (ii) decrease the speed of the vacuum pump 132 when the determined outlet airflow rate exceeds the defined outlet airflow rate.

In some embodiments, a pressure within the outlet air tube 140 may be indicative of system operation. For example, an elevated pressure within the outlet air tube 140 during operation of the vacuum pump 132 may indicate a blockage along the outlet air tube 140, such as a clogged charcoal filter 146, for example. In some embodiments, the logic 221 may compare an outlet pressure as determined by the outlet pressure sensor 144 with an outlet pressure limit stored in the memory 220, and based on the comparison, the logic 221 may provide a notification to the clinician when the determined outlet pressure exceeds the outlet pressure limit.

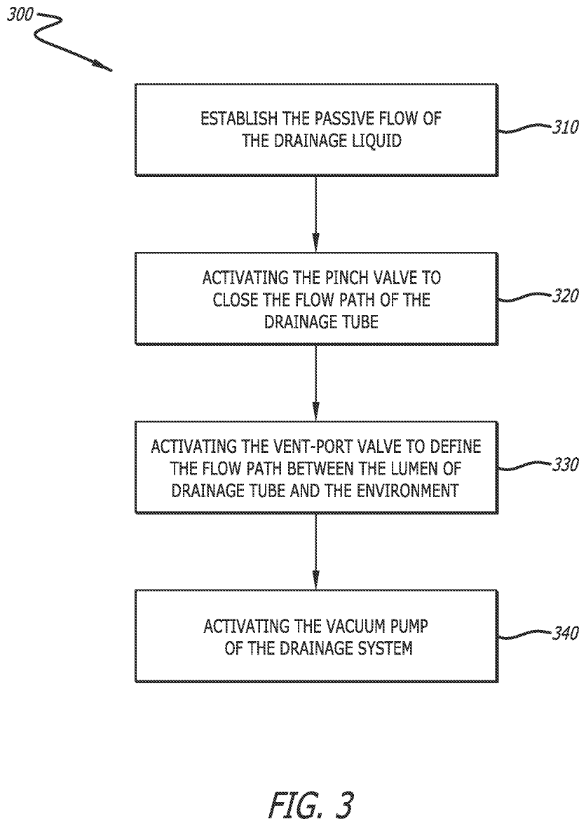

FIG. 3 illustrates a block diagram of a system method 300 for draining liquid from a patient that, according to some embodiments, includes all or any subset of the following actions, steps, or processes. The method 300 may establish the passive flow of the drainage liquid from the patient along the drainage tube of the drainage system from the catheter to the collection container (block 310). In some embodiments, establishing the passive flow includes transitioning the pinch valve to the open state and/or transitioning the vent-port valve to the closed state.

The method 300 may further include activating the pinch valve to close the flow path of the drainage tube between the vent-port valve and the catheter (block 320).

The method 300 may further include activating the vent-port valve to define the flow path between the lumen of drainage tube and the environment (block 330). In some embodiments of the method, activating the vent-port valve occurs after activating the vacuum pump to cause a buildup of vacuum within the collection container, such that subsequently activating the vent-port valve causes a surge of airflow proximally along the drainage tube.

The method 300 may further include activating the vacuum pump of the drainage system (block 340) to generate the vacuum within the collection container, thereby defining the airflow proximally along the drainage tube from the vent-port valve to the collection container.

In some embodiments, the method 300 may further include determining the vacuum level within the collection container via the pressure sensor. In some embodiments, the method 300 further includes (i) comparing the vacuum level with a vacuum level limit stored in the memory and (ii) adjusting the speed of the vacuum pump based on the comparison of the vacuum level with the vacuum level limit.

In some embodiments, the method 300 may further include (i) comparing the vacuum level with a safe vacuum limit stored in the memory and (ii) deactivating the pinch valve only when vacuum level is below the safe vacuum limit.

In some embodiments of the method 300, the system includes the vacuum relief valve fluidly coupled with the air inlet tube, where the vacuum relief valve defines a vacuum relief limit, and where (i) the vacuum relief valve remains in a default closed state when the vacuum level is below the vacuum relief limit and (ii) the vacuum relief valve self-transitions from the default closed state to an open state to relieve the vacuum within the collection container when the vacuum level exceeds the vacuum relief limit.

In some embodiments, the vacuum relief valve is an electro-mechanical relief valve, and the method 300 further includes (i) comparing the vacuum level with the vacuum relief limit stored in the memory and (ii) activating the vacuum relief valve to transition the vacuum relief valve from the default closed state to the open state to relieve the vacuum within the collection container when the vacuum level exceeds a vacuum relief limit stored in the memory.

In some embodiments, the system further includes the inlet flow meter coupled in line with the air inlet tube, and the method 300 further includes determining the inlet airflow rate within the inlet air tube when the vacuum pump is activated.

In some embodiments, the method 300 further includes (i) comparing the inlet airflow rate with a predefined first airflow rate stored in the memory and (ii) adjusting the speed of the vacuum pump based on the comparison of the inlet airflow rate with the predefined first airflow rate.

In some embodiments, the system further includes the outlet flow meter coupled in line with an air outlet tube of the vacuum pump, and the method 300 further includes determining the outlet airflow rate within the outlet air tube when the vacuum pump is activated. In some embodiments, the method further includes (i) comparing the outlet airflow rate with a predefined outlet airflow rate stored in the memory and (ii) adjusting the speed of the vacuum pump based on the comparison of the outlet airflow rate with the predefined outlet airflow rate.

In some embodiments, the system further includes a fluid detector operatively coupled with the drainage tube, and the method 300 further includes (i) detecting the liquid within the drainage tube and (ii) activating the vacuum pump in response detecting the liquid within the drainage tube.

In some embodiments, the system further includes the outlet pressure sensor fluidly coupled with the air outlet tube, and the method 300 further includes (i) determining the outlet pressure within the outlet tube, (ii) comparing the outlet pressure with an outlet pressure limit stored in the memory, and (iii) providing a notification when the outlet pressure exceeds the outlet pressure limit.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A drainage system for draining a liquid from a patient, the drainage system comprising:
a drainage tube defining a proximal end and a distal end, the drainage tube fluidly coupled with a catheter so as to receive a drainage liquid from the catheter,
a vent-port valve operatively coupled to the drainage tube adjacent the distal end, the vent-port valve transitionable between:
a first valve state defining a closed flow path extending between a lumen of the drainage tube and the environment, and
a second valve state defining an open flow path extending between a lumen of the drainage tube and the environment;
a pinch valve operatively coupled with the drainage tube between the vent-port valve and the catheter, the pinch valve transitionable between:
an open state defining a flow path along the drainage tube between the catheter and the vent-port valve, and
a closed state defining a closure of the flow path along the drainage tube between the catheter and the vent-port valve;
a collection container coupled with the drainage tube at the proximal end, the collection container configured to collect the drainage liquid;
a vacuum pump fluidly coupled with the collection container via an air inlet tube;
a fluid detector operatively coupled with the drainage tube, the fluid detector configured to detect the presence of the drainage liquid within the drainage tube, and the fluid detector configured to operatively couple with the drainage tube at any location along the drainage tube; and
system module operatively coupled with the vacuum pump, the fluid detector, and the pinch valve, the system module including a console having a processor and a memory with logic stored thereon that, when executed by the processor, causes operations of the drainage system that include:
activating the pinch valve to transition the pinch from the open state to the closed state; and
activating the vacuum pump in response to the fluid detector detecting the presence of the drainage liquid within the drainage tube to generate a vacuum within collection container such that when the vent-port valve is transitioned from the first valve state to the second valve state, the vacuum within the collection container causes an airflow proximally along the drainage tube from the vent-port valve to the collection container.

2. The system according to claim 1, wherein:
the vent-port valve is an electro-mechanical vent-port valve operatively coupled with the system module, and
the operations include activating the vent-port valve to transition the vent-port valve from the first state to the second valve state.

3. The system according to claim 2, wherein the operations further include:
activating the vacuum pump with the vent-port valve disposed in the first valve state and the pinch valve disposed in the closed state to cause a buildup of vacuum within the collection container, and
activating the vent-port valve to release the buildup of vacuum within the collection container, thereby causing a surge of airflow proximally along the drainage tube.

4. The system according to claim 1, further comprising a pressure sensor fluidly coupled with the air inlet tube and operatively coupled with the system module, the pressure sensor configured to determine a vacuum level within the collection container.

5. The system according to claim 1, wherein the operations further include:
comparing the vacuum level with a vacuum level limit stored in the memory; and
adjusting a speed of the vacuum pump based on the comparison of the vacuum level with the vacuum level limit.

6. The system according to claim 4, wherein the operations further include:
comparing the vacuum level with a safe vacuum limit stored in the memory; and
deactivating the pinch valve to transition the pinch valve from the closed state to the open state only when the vacuum level is below the safe vacuum limit.

7. The system according to claim 1, further comprising a vacuum relief valve fluidly coupled with the air inlet tube, the vacuum relief valve defining a vacuum relief limit, wherein:
the vacuum relief valve remains in a default closed state when the vacuum level is below the vacuum relief limit, and
the vacuum relief valve self-transitions from the default closed state to an open state to relieve vacuum within the collection container when the vacuum level exceeds the vacuum relief limit.

8. The system according to claim 1, further comprising an electro-mechanical vacuum relief valve fluidly coupled with the air inlet tube and operatively coupled with the system module, the operations include:
comparing the vacuum level with a vacuum relief limit stored in the memory; and
activating the electro-mechanical vacuum relief valve to transition the vacuum relief valve from the default closed state to the open state to relieve the vacuum within the collection container when the vacuum level exceeds a vacuum relief limit stored in the memory.

9. The system according to claim 1, further comprising a first flow meter coupled in line with the air inlet tube and operatively coupled with the system module, the first flow meter configured to determine a first airflow rate within the inlet air tube when the vacuum pump is activated.

10. The system according to claim 9, wherein the operations further include:
comparing the first airflow rate with a predefined first airflow rate stored in the memory; and
adjusting a speed of the vacuum pump based on the comparison of the first airflow rate with the predefined first airflow rate.

11. The system according to claim 1, further comprising a second flow meter coupled in line with an air outlet tube of the vacuum pump and operatively coupled with the system module, the second flow meter configured to determine a second airflow rate within the outlet air tube when the vacuum pump is activated.

12. The system according to claim 11, wherein the operations further include:
comparing the second airflow rate with a predefined second airflow rate stored in the memory; and
adjusting a speed of the vacuum pump based on the comparison of the second airflow rate with the predefined second airflow rate.

13-14. (canceled)

15. The system according to claim 1, further comprising a charcoal filter disposed in line with outlet air tube such that air exiting the outlet tube passes through the charcoal filter.

16. The system according to claim 1, further comprising an outlet pressure sensor fluidly coupled with the air outlet tube and operatively coupled with the system module, the outlet pressure sensor configured to determine an outlet pressure within the outlet tube.

17. The system according to claim 16, wherein the operations further include:
comparing the outlet pressure with an outlet pressure limit stored in the memory; and
providing a notification when the outlet pressure exceeds the outlet pressure limit.

18-31. (canceled)
